(19) United States
(12) Patent Application Publication
(10) Pub. No.: US 2011/0295324 A1
(43) Pub. Date: Dec. 1, 2011

(54) TABBED COMPRESSION PLATE AND METHOD OF USE

(76) Inventors: Brian Donley, Solon, OH (US); Luis Arellano, Fair Lawn, NJ (US); Jamy Gannoe, West Milford, NJ (US); Jeff Tyber, Bethlehem, PA (US)

(21) Appl. No.: 13/052,863

(22) Filed: Mar. 21, 2011

Related U.S. Application Data
(60) Provisional application No. 61/340,613, filed on Mar. 19, 2010.

(51) Int. Cl. A61B 17/80

(52) U.S. Cl. 606/289

(57) ABSTRACT

An orthopedic fixation system includes a compression plate member, a lag screw member, and a plurality of threaded screw members for applying compression across a fracture site. The compression plate member has a generally planar body, an upper surface, and bone contacting surface. The planar body includes a plurality of through-apertures and a hinged tab member having an elongated through aperture, which is adapted to receive the lag screw member and cause the tab member to recess towards a bone divot formed in the underlying bone. The compression plate member is also adapted to receive the plurality of screw members within the through-apertures for fixably and threadably coupling the compression plate member across a fracture site. The lag screw member applies a direct compressive force across the fracture site through the deformable tab that is recessed in the bone divot.

### CROSS-REFERENCE TO RELATED APPLICATIONS

This non-provisional application claims the benefit of provisional application No. 61/340,613, filed Mar. 19, 2010, which is herein incorporated by reference in its entirety.

### FIELD OF THE INVENTION

This invention relates to the field of orthopedic implant devices and, particularly, to an orthopedic compression plate and screw assembly for providing a direct compressive force across a fracture site to secure two or more bone fragments or bones together.

### BACKGROUND OF THE INVENTION

Orthopedic implant devices are often used to repair or reconstruct bones and joints caused by bone fractures, degenerative bone conditions, or other similar types of injuries. Frequently, these orthopedic devices require that bone fragments, due to bone fractures or bones cut by a surgical operation (i.e., an osteotomy), must be kept together for long periods of time or short periods intraoperatively under a sustained force across the fractures site in order to promote healing and stabilizing the bone fragments. As such, these orthopedic implant devices have several functions. These devices may be used to realign bone segments, to apply interfragmental compression to bone fragments, or to restore native geometries.

For example, most orthopedic implants are constructed from one-piece or two-piece members and comprise threaded screws for attaching these implant devices to bone fragments. In addition, these orthopedic implant devices are constructed from standard materials, which undergo normal elastic-plastic mechanical responses during tightening. These orthopedic implants apply initial interfragmental compression, however, due to the biological conditions of bone resorbtion (i.e., removal of bone), which may be sometimes caused from micromotion across lines of fracture, interfragmental compression is lost as implants loosen due to the resorbtion of fragmental contacting surfaces, thereby causing the fragments or device to shorten. This biological condition eliminates ideal conditions for bone healing, as stated by Wolff's law: bone grows under load and resorbs (i.e. removed) in the lack of loads. Thus, these orthopedic implant devices are not very effective in maintaining interfragmental compression for long periods as is required in order to heal the fracture site.

Other devices utilize deformable compression staples or deformable compression plate and screw constructs that provide compression by using a tool to deform and shorten the length of the compression staples or plates. Yet, these too are inefficient, as these staple or plate constructs do not apply the required compression across the fracture site needed to hold and compress the bone fragments. In addition, implant loosening is a serious concern and is commonly caused by one or multiple conditions, such as subsidence, centering, fixation loosening or cortical failure to name a few.

There is, therefore, a need for an orthopedic implant device assembly and a method of use for the orthopedic implant device assembly that overcomes some or all of the previously delineated drawbacks of prior orthopedic implant device assemblies.

### SUMMARY OF THE INVENTION

An object of the invention is to overcome these and other drawbacks of previous inventions.

Another object of the invention is to provide a novel and useful orthopedic fixation assembly that may be utilized to secure multiple bones fragments or bones together.

Another object of the invention is to provide an orthopedic fixation assembly that may be utilized to secure the implant bone interface.

Another object of the invention is to provide an orthopedic fixation assembly for facilitating direct compression across the fracture site.

Another object of the invention is to provide an orthopedic fixation assembly having a plate and screw construct for preventing rotation of the adjacent constructs.

Another object of the invention is to provide a hybrid orthopedic fixation assembly for applying direct compression through a hinge-like mechanism.

Another object of the invention is to provide a fixation assembly having a hybrid plate member that accepts a variable angle screw in order to provide a compound variable angle construct.

In a first non-limiting embodiment of the invention, a compression plate for orthopedic fixation is provided and includes a coplanar body portion having an upper surface and a directly opposed bone contacting surface. The body portion includes a longitudinal axis. The compression plate has a plurality of bone screw holes extending orthogonally through the upper and bone contacting surfaces, with each of the bone screw holes configured for receiving a bone screw. Finally, the compression plate has a generally rectangular tab member residing within an elongated slot in the body portion.

In a second non-limiting embodiment of the invention, an orthopedic fixation system for bone fusion includes a compression plate having a body portion, with the body portion having an upper surface, a directly opposed bone contacting surface, a plurality of bone screw holes, and a generally rectangular tab member residing within an elongated slot in said body portion. The body portion has a first section defining a first longitudinal axis, and a second section defining a second longitudinal axis. The plurality of bone screw holes extend orthogonally through the upper and bone contacting surfaces. The system further includes a plurality of threaded bone screws that are configured to be received in the compression plate.

In a third non-limiting embodiment of the invention, an orthopedic fixation assembly is provided and includes a compression plate member, a lag screw member, and a plurality of threaded screw members for applying compression across a fracture site. The compression plate member has a generally planar body and a first exposed surface and a second opposed surface. The planar body includes a plurality of through apertures and a hinged tab member having an elongated through aperture, which is provided to receive the lag screw member. The tab member recesses in a bone divot formed in the underlying bone. The compression plate member receives the plurality of screw members within the through apertures in order to fixably couple the compression plate member across a fracture site. The lag screw member applies a direct compressive force across the fracture site through the deformable tab being recessed in the bone divot.

In a fourth non-limiting embodiment of the invention, a method for inserting an orthopedic fixation assembly into bone fragments is provided and includes several non-limiting steps. In one step, a Kirschner wire inserted at a desired trajectory angle into the human foot. The Kirschner wire is coupled to a standard drill and inserted into the calcaneus bone and cuboid bone at a desired trajectory, which represents the desired trajectory of a lag screw member. The position of the inserted Kirschner wire may be verified through fluoroscopy and its position inside cuboid bone may be adjusted so that the tapered end of Kirschner wire resides at a desired depth. Next, in another step, the Kirschner wire is coupled to a cannulated drill and a pilot hole is drilled into the cuboid bone to a desired depth at predetermined trajectory of the Kirschner wire. The depth of the pilot hole is determined based on the desired length of the lag screw. Next, another step, a cannulated countersink drill is inserted over the Kirschner wire and drilled into the surface of calcaneus bone in order to create a bone divot for a hinged tab member. The recommended depth of bone divot is determined by marking the countersink drill and drilling into the surface of the calcaneus bone to this depth. The Kirschner wire is removed from the cuboid and calcaneus bones after countersinking. Next, in another step, a threaded screw member is inserted into the compression plate member in one side of the joint or fracture site. The compression plate member may be selected so that the desired length of the plate member will span across the fusion site and leave an adequate length between the opposed threaded screws. A pilot hole is predrilled into the bone and a threaded screw member is inserted into the pilot hole. The threaded screw member provides retention of the compression plate member into bone and locks the compression plate member for receiving the other threaded screw members. Next, in another step, lag screw member is inserted into the elongated aperture of compression plate member through the created trajectory. The lag screw member will deform the tab member towards the surface of the calcaneus bone and the tab will recede into the bone divot while the lag screw member is driven across the joint and compression is established. The lag screw member is driven into the joint until satisfactory compression is achieved. The position of the inserted lag screw member may be verified through fluoroscopy and its position inside joint may be adjusted so that the lag screw member resides at a desired depth. Next, in another step, pilot holes are predrilled into the unused apertures of the compression plate member and the remaining threaded screw members are inserted into these holes in order to threadably couple the compression plate member to the bone. The position of the inserted screw members may be verified through fluoroscopy.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of an orthopedic fixation assembly inserted into the bones of a patient's foot according to the preferred embodiment of the invention.

FIG. 2 is another perspective view of the orthopedic fixation assembly that was shown in FIG. 1.

FIG. 3 is a perspective view of the hinged tab member, which was shown in FIGS. 1 and 2 according to the preferred embodiment of the invention.

FIG. 4 illustrates a surgical step of inserting the orthopedic fixation assembly of FIG. 1 using a Kirschner wire according to the preferred embodiment of the invention.

FIG. 5 illustrates another surgical step of installing the orthopedic fixation assembly of FIG. 1 using a countersink drill according to the preferred embodiment of the invention.

FIG. 6 illustrates another surgical step of installing the orthopedic fixation assembly of FIG. 1 using the screw members according to the preferred embodiment of the invention.

FIG. 7 is a perspective view of the assembled orthopedic fixation assembly inserted into the calcaneus and cuboid bones of a patient's foot according to an embodiment of the invention.

FIG. 8 is a flow chart illustrating the surgical method of coupling the orthopedic fixation assembly shown in FIGS. 1-7 to the calcaneus and cuboid bones in a patient's foot according to an embodiment of the invention.

### DETAILED DESCRIPTION OF THE INVENTION

The invention may be understood more readily by reference to the following detailed description of preferred embodiment of the invention. However, techniques, systems, and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

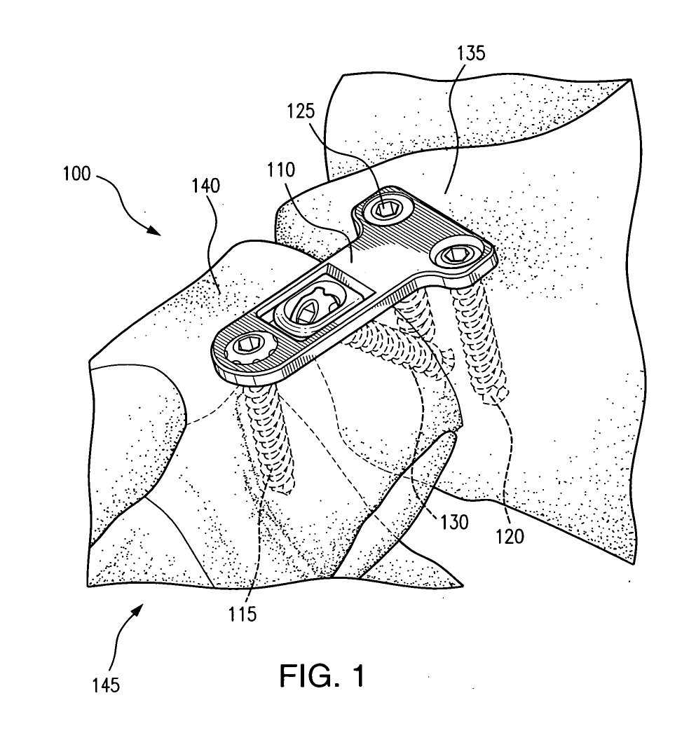

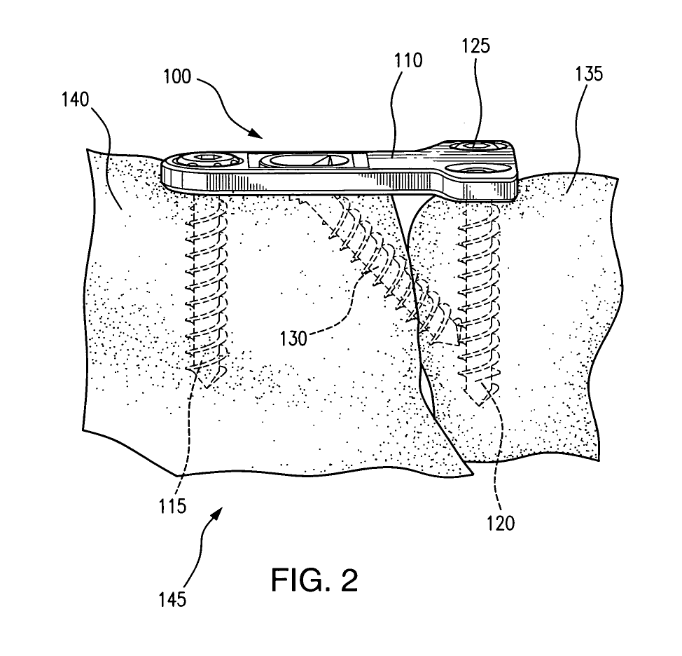

Referring now to FIGS. 1-2, there is shown an orthopedic fixation assembly 100 which is made in accordance with the teachings of the preferred embodiment of the invention. As shown, orthopedic fixation assembly 100 comprises a generally coplanar compression plate member 110, which is provide to selectively receive a plurality of threaded screws 115, 120, 125, and a threaded lag screw 130. In the preferred embodiment, threaded lag screw 130 is a variable angle screw. The lag screw 130 is received in plate member 110 and cooperates with compression plate member 110 in order to selectively apply compression across the bone fracture site in the human foot 145. In another non-limiting embodiment, the threaded lag screw 130 may be a fixed angle screw incorporating a Morse taper lock. Also, threaded screws 115, 120, and 125 may be fixed angle screws, variable angle screws, or a combination of fixed and variable angle screws depending on the needs of a surgeon. It should be appreciated that the orthopedic fixation assembly 100 is provided to be inserted across any bone or through a plurality of bones, such as in one non-limiting example, the calcaneus bone 135 and the cuboid bone 140 in the human foot 145, although in other embodiments, the orthopedic fixation assembly 100 is provided to be inserted into substantially any other bones or parts of bones. It should also be appreciated that the orthopedic fixation assembly 100 may be utilized for the reconstruction and fusion of joints of the extremities in order to apply direct and evenly distributed compression across the joint or fracture site or on the bones in the foot 145.

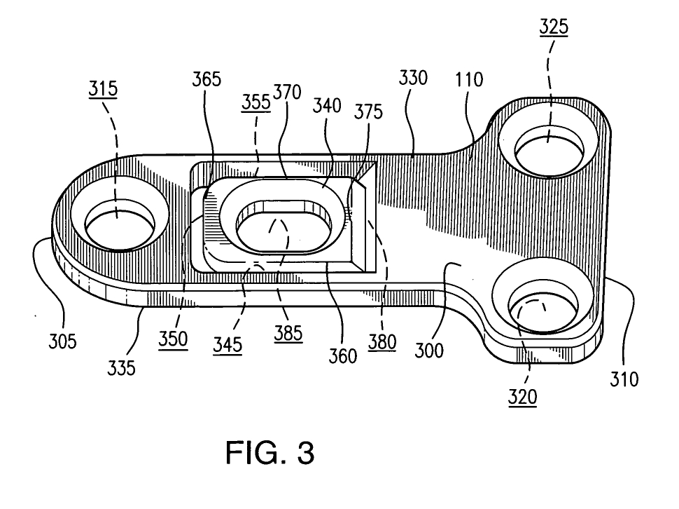

Also, as shown in FIG. 3, compression plate member 110 has a generally coplanar body 300 from a first end 305 to a second end 310 (i.e., body 300 has a constant thickness from first end 305 to second end 310). Body 300 includes a plurality of transverse apertures 315, 320, and 325, which are provided at first end 305 and second end 310 respectively (i.e., aperture 315 is provided at first end 305 and apertures 320, 325 are provided at second 310). The plurality of apertures 315, 320, and 325 traverse the surfaces of body 300 (i.e., penetrate body 300) from first surface 330 to opposed second surface 335. The apertures 315, 320, and 325 are provided to receive a plurality of screws 115, 120, and 125 respectively (shown in FIGS. 1-2) in order to couple the compression plate member 110 to the bones in the human foot 145 (shown in FIGS. 1-2) or other similar bones. Threaded screws 115, 120, and 125 may be fixed angle screws, variable angle screws, or a combination of fixed and variable angle screws depending on the needs of a surgeon. In other non-limiting embodiments, variable angle screws or locking fixed or variable angle screws having a Morse taper lock between the screw head and the apertures 315, 320, and 325 may be utilized for any of the screws 115, 120, and 125.

Additionally, compression plate member 110 has a hinged tab member 340 formed in body 300. Particularly, hinged tab member 340 is generally rectangular in shape and includes a plurality of channels 345, 350, and 355 formed along the three edges of tab member 340. Particularly, tab member 340 has channel 345 formed along edge 360, channel 350 formed along edge 365, and channel 355 formed along edge 370. Fourth edge 375 includes a hinge formed in groove 380 recessed along length of edge 375, hingedly coupled to plate member 110, and generally coextensive with length of edge 375. Channels 345, 350, and 355 cooperate with hinge to cause tab member 340 to bend (or flex) along the hinge formed by groove 380 along edge 375 and body 300, at a multitude of angles upon application of force on tab member 340. It should be appreciated that tab member 340 cooperates with a variable angle lag screw 130 (shown in FIGS. 1-2) to provide a compound variable angle for positioning the tab member 340 on the bone surface, causing the lag screw 130 to provide compression across the fracture site or joint while the plate member 110 maintains the compressed position of the bones.

Also, hinged tab member 340 includes aperture 385 for receiving a threaded lag screw 130 (shown in FIGS. 1-2). The aperture 385 is generally elongated in shape and traverses the surface of body 300 (i.e., penetrates body 300) from first surface 330 to opposed second surface 335. The aperture 385, being elongated, allows for various 385 or variable angle screws to be inserted into aperture 385 at various angles of fixation. It should be appreciated that aperture 385 is provided to cooperate with lag screw 130 (shown in FIGS. 1-2) to deform tab member 340 towards the surface of the underlying bone, thereby facilitating application of compression across the joint or fracture site. It should also be appreciated that the tab member 340 may recess into a dimple created on the underlying bone surface to facilitate additional purchase of the lag screw 130 into bone. It should also be appreciated second surface 335 of plate member 110 may be coated with an osteoconductive material, such as, for example, plasma spray or other similar types of porous materials that is capable of supporting or encouraging bone ingrowth into this material.

In operation, and as best shown in FIGS. 1 and 4-8, the orthopedic fixation assembly 100 may be utilized for osteotomies and arthrodeses of the foot 145 by connecting and compressing the damaged bones in order to promote healing. In other non-limiting embodiments, the orthopedic fixation assembly 100 may also be utilized to apply compression to the other bones in the human body. In one example shown in FIG. 1, the orthopedic fixation assembly 100 may be coupled to the calcaneus bone 135 and the cuboid bone 140 in order to provide direct compression and stability across the fracture site of the joint connecting the calcaneus bone 135 to the cuboid bone 140.

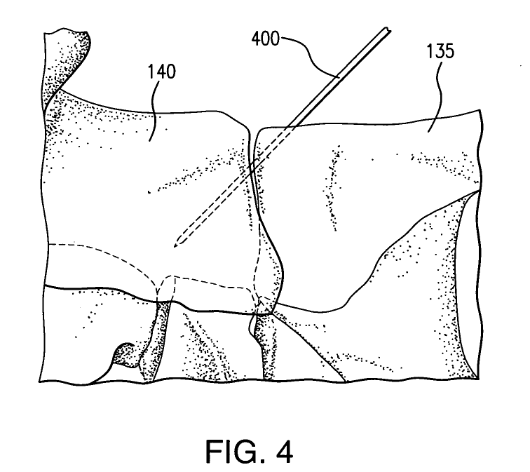

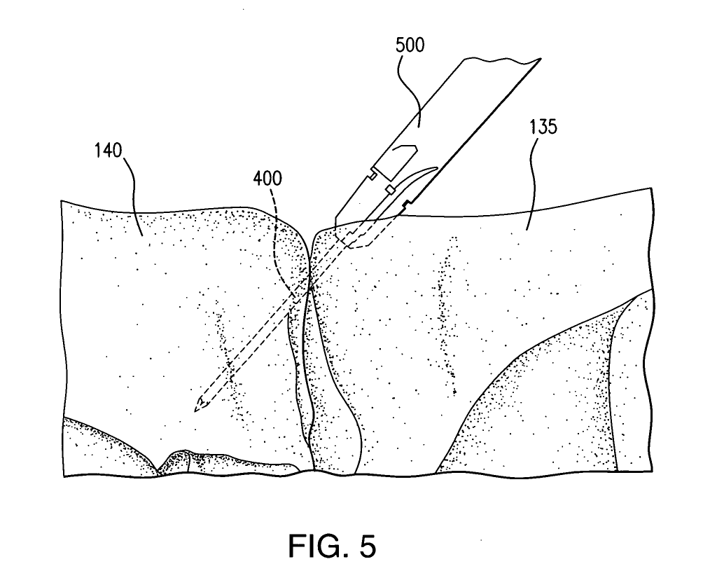

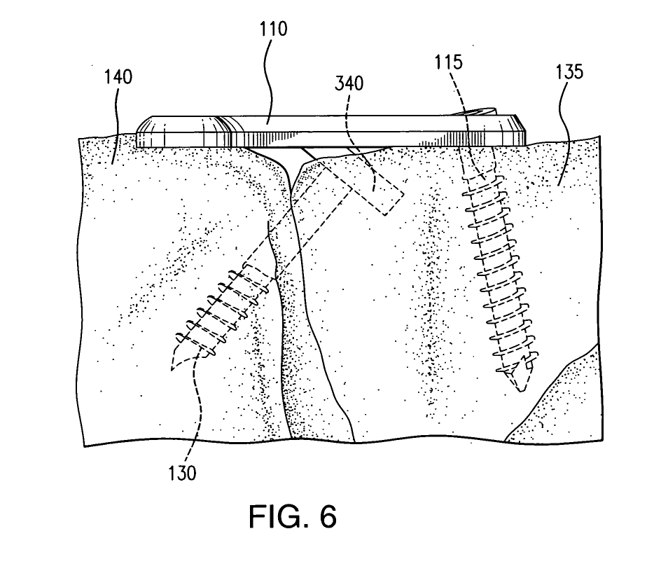

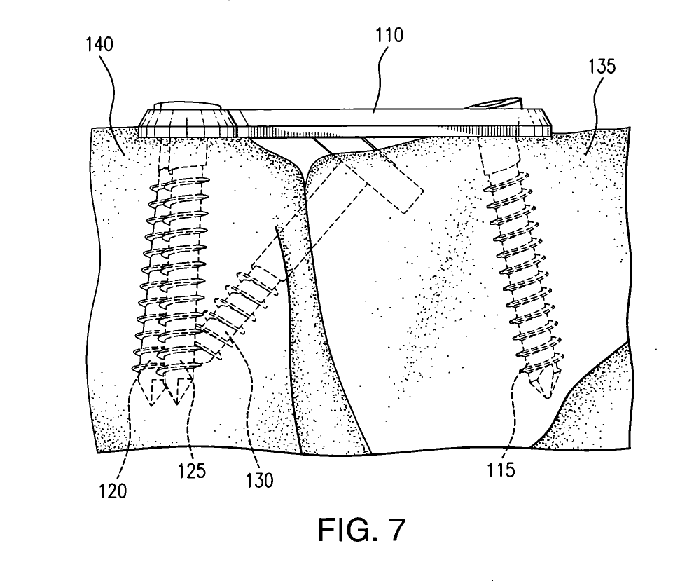

As shown in FIGS. 4-8, the orthopedic fixation assembly 100 may be utilized for, in one non-limiting embodiment, the internal fixation of bone or bone fragments in the human foot 145 (FIG. 1). As shown, the method starts in step 800 and proceeds to step 802, whereby a Kirschner wire 400 is inserted at a desired trajectory angle into the human foot 145 (FIG. 1). In this step, a Kirschner wire 400 is selected and coupled to a standard drill (not shown) and inserted into the calcaneus bone 135 and cuboid bone 140 at a desired trajectory, which represents the desired trajectory of the lag screw 130 (FIG. 1). The position of the inserted Kirschner wire 400 may be verified through fluoroscopy and its position inside cuboid bone 140 may be adjusted so that the tapered end of Kirschner wire 400 resides at a desired depth. Next, in step 804, the Kirschner wire 400 is coupled to a cannulated drill and a pilot hole is drilled into the cuboid bone 140 (FIG. 4) to a desired depth at predetermined trajectory of the Kirschner wire 400. The depth of the pilot hole is determined based on the desired length of the lag screw 130 (shown in FIG. 1). Next, in step 806, a cannulated countersink drill 500 (shown in FIG. 5) is inserted over the Kirschner wire 400 and drilled into the surface of calcaneus bone 140 in order to create a bone divot for hinged tab member 340 (shown in FIG. 3). The recommended depth of bone divot is determined by marking the countersink drill 500 and drilling into the surface of the calcaneus bone 135 to this depth. The Kirschner wire 400 is removed from the bones 135 and 140 after countersinking. Next in step 808, threaded screw member 115 is inserted into the compression plate member 110 in one side of the joint or fracture site. The compression plate member 110 (shown in FIG. 6) may be selected so that the desired length of the plate member 110 will span across the fusion site and leave an adequate length between the opposed threaded screws. A pilot hole is predrilled into aperture 315 (FIG. 3) and a threaded screw member 115 is inserted into the aperture 315 and into the pilot hole. The threaded screw member 115 provides retention of the compression plate member 110 into bone 135 and locks the compression plate member 110 for receiving the other threaded screw members. Next, in step 810, lag screw member 130 is inserted into the elongated aperture 385 (FIG. 3) of compression plate member 110 through the created trajectory. The lag screw member 110 will deform the tab member 340 towards the surface of the calcaneus bone 135 and the tab will receded into the bone divot while the lag screw member 130 is driven across the joint and compression is established. The lag screw member 130 is driven into the joint until satisfactory compression is achieved. The position of the inserted lag screw member 130 may be verified through fluoroscopy and its position inside joint may be adjusted so that the lag screw member 130 resides at a desired depth. Next, in step 812, pilot holes are predrilled into apertures 320 and 325 (shown in FIG. 3) and the remaining threaded screw members 120, 125 (shown in FIG. 7) are inserted into their respective holes 320, 325 (FIG. 3) in order to threadably couple the compression plate member 110 to the cuboid bone 140 (Shown in FIG. 7). The position of the inserted screw members 120, 125 may be verified through fluoroscopy. The method ends in step 814.

It should also be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. A compression plate for orthopedic fixation, comprising:
a coplanar body portion including an upper surface and a directly opposed bone contacting surface, wherein said body portion includes a first section defining a first longitudinal axis, and a second section defining a second longitudinal axis;
a plurality of bone screw holes extending orthogonally through said upper and bone contacting surfaces, each of said bone screw holes configured for receiving a bone screw; and
a generally rectangular tab member residing within an elongated slot in said body portion.

2. The compression plate of claim 1, wherein said first longitudinal axis is orthogonal to said second longitudinal axis, wherein said first and said second longitudinal axes reside in a common plane.

3. The compression plate of claim 1, wherein said first section includes a first end having a first bone screw hole and a directly opposed second end that is connected to a mid-point of said second section.

4. The compression plate of claim 1, wherein said second section includes at least a second and a third bone screw hole.

5. The compression plate of claim 1, wherein said elongated slot is disposed within said first section and includes a length that is aligned longitudinally along the first longitudinal axis.

6. The compression plate of claim 1, wherein said tab member is coupled to said first section along one edge.

7. The compression plate of claim 6, wherein said one edge is parallel to said second longitudinal axis along an edge that is furthest away from said first end.

8. The compression plate of claim 1, wherein said tab member includes an elongated screw hole aligned along a hole axis that is orthogonal to said upper and said bone contacting surfaces.

9. The compression plate of claim 8, wherein said elongated screw hole is configured for receiving a polyaxial threaded screw.

10. The compression plate of claim 6, wherein said tab member is configured for pivoting along said one edge.

11. The compression plate of claim 6, wherein said tab member is located substantially near a midpoint of said first section.

12. An orthopedic fixation system for bone fusion, comprising:
a compression plate having a body portion, said body portion includes an upper surface, a directly opposed bone contacting surface, a plurality of bone screw holes, and a generally rectangular tab member residing within an elongated slot in said body portion; and
a plurality of threaded bone screws configured to be received in said compression plate;
wherein said body portion includes a first section defining a first longitudinal axis, and a second section defining a second longitudinal axis;
wherein said plurality of bone screw holes extend orthogonally through said upper and bone contacting surfaces.

13. The fixation system of claim 12, wherein said first longitudinal axis is orthogonal to said second longitudinal axis, and wherein said first and said second longitudinal axis reside on a common plane.

14. The fixation system of claim 12, wherein said first section includes a first end having a first bone screw hole and an opposed second end that is connected at a mid-point of said second section.

15. The fixation system of claim 12, wherein said second section includes at least a second and a third bone screw hole.

16. The fixation system of claim 12, wherein said elongated slot is disposed within said first section and includes a length that is aligned longitudinally along the first longitudinal axis.

17. The fixation system of claim 12, wherein said tab member is coupled to said first section along one edge.

18. The fixation system of claim 17, wherein said one edge is parallel to said second longitudinal axis along an edge that is furthest away from said first end.

19. The fixation system of claim 12, wherein said tab member includes an elongated screw hole aligned along a hole axis that is orthogonal to said upper and said bone contacting surfaces.

20. The fixation system of claim 19, wherein said body portion is configured for receiving at least one polyaxial locking screw.

21. The fixation system of claim 17, wherein said tab member is configured for deforming away from said upper surface along said one edge and for deforming towards said upper surface along said one edge.

22. The fixation system of claim 17, wherein said tab member is located substantially near a midpoint of said first section.

23. A method for bone fusion, comprising the steps of:
providing a compression plate having a coplanar body portion with an upper surface, a directly opposed bone contacting surface, and a plurality of bone screw holes extending orthogonally through the upper and bone contacting surfaces;
forming a first medullary canal in a first bone along an axis orthogonal to the upper and contacting surfaces and aligned with a first bone screw hole;
forming a second and third medullary canal in a second bone with each being aligned along an axis orthogonal to the upper and contacting surfaces, wherein the second medullary canal is aligned with a second bone screw hole and the third medullary canal is aligned with a third bone screw hole;
forming a divot on an exposed surface of the first bone;
forming a third medullary canal through the divot along a predetermined trajectory;
inserting a first threaded screw into the first medullary canal and inserting a second and third threaded screw into each of the second and third medullary canals;
inserting a threaded lag screw into the third medullary canal along the predetermined trajectory; and
compressing the underlying joint spanning the first and the second bone.

24. The method of claim 23, further comprising inserting the threaded lag screw into an elongated screw hole located on a generally rectangular tab member in the compression plate.

25. The method of claim 24, wherein the body portion includes a first section defining a first longitudinal axis, and a second section defining a second longitudinal axis.

26. The method of claim 25, wherein the first longitudinal axis is orthogonal to the second longitudinal axis, wherein the first and the second longitudinal axis reside in a common plane.

27. The method of claim 25, wherein the first section includes the first bone screw hole positioned at a first end and an opposed second end that is connected at a mid-point of the second section.

28. The method of claim 25, wherein the second section includes the second and the third bone screw holes directly opposed to each other.

29. The method of claim 25, wherein the elongated slot is disposed within the first section and includes a length that is aligned longitudinally along the first longitudinal axis.

30. The method of claim 27, wherein the tab member is coupled to the first section along one edge.

31. The method of claim 30, wherein the one edge is parallel to the second longitudinal axis along an edge that is furthest away from the first end.

32. The method of claim 25, wherein the elongated screw hole is aligned along a hole axis that is orthogonal to the upper and the bone contacting surfaces.

33. The method of claim 23, further comprising inserting at least one polyaxial locking screw into the first, second, and third medullary canals.

34. The method of claim 30, further comprising deforming the tab member away toward the first bone along the one edge.
